(19) United States
(12) Patent Application Publication
(10) Pub. No.: US 2003/0013688 A1
(43) Pub. Date: Jan. 16, 2003

(54) Synergistic combination of pravastatin and aspirin and method

(76) Inventors: Rene Belder, Hopewell, NJ (US); Kannan Natarajan, Newtown, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/128,621

(22) Filed: Apr. 23, 2002

Related U.S. Application Data
(60) Provisional application No. 60/299,959, filed on Jun. 21, 2001.

(51) Int. Cl. A61K 31/366; A61K 31/225

(52) U.S. Cl. 514/161; 514/163; 514/548; 514/460

(57) ABSTRACT

A synergistic combination of pravastatin and aspirin is provided which is formed of 40 mg pravastatin and 81 mg or higher of aspirin, preferably 81 mg aspirin or 325 mg of aspirin. A method for preventing, inhibiting or reducing risk of onset of cardiovascular events or cerebrovascular events employing such synergistic combination is also provided.

This application claims priority from U.S. Provisional Application No. 60/299,959 filed Jun. 21, 2001, the entirety of which is incorporated herein by reference.

### BACKGROUND OF THE INVENTION

The use of aspirin for reducing the risk of a myocardial infarction and the use of statins for lowering cholesterol and preventing or treating atherosclerosis and cardiovascular disease and cerebrovascular disease are well documented. In fact, it is not uncommon that patients having elevated cholesterol levels who are at high risk for a myocardial infarction take both a statin and aspirin.

U.S. Pat. No. 6,235,311 to Ullah et al discloses a combination of a statin including pravastatin and aspirin for preventing, reducing and/or treating elevated cholesterol levels, atherosclerosis, cardiovascular events and disease including coronary events and cerebrovascular events, and coronary artery disease and/or cerebrovascular disease. Ullah et al teach use of pravastatin in amounts from about 10 to about 80 mg and aspirin in amounts from about 10 to about 800 mg.

U.S. Pat. No. 5,674,893 to Behounek et al discloses a method for preventing or reducing risk of cardiovascular events employing a statin including pravastatin.

U.S. Pat. No. 5,622,985 to Olukotun et al discloses a method for preventing a second heart attack employing an HMG CoA reductase inhibitor including pravastatin.

### DESCRIPTION OF THE INVENTION

In accordance with the present invention, a synergistic combination of pravastatin and aspirin is provided. In preferred embodiments, the pravastatin and aspirin will be in a weight ratio of about 1:2 or higher. Preferred combinations will include 40 mg pravastatin and 81 mg or higher aspirin. Most preferred are combinations of 40 mg pravastatin and 81 mg aspirin, and 40 mg pravastatin and 325 mg aspirin.

In addition, in accordance with the present invention, a pharmaceutical formulation is provided which includes a synergistic combination of pravastatin and aspirin as described above, and a pharmaceutically acceptable carrier therefor.

Further, in accordance with the present invention, a method is provided for preventing, inhibiting or reducing the risk of onset of a cardiovascular event or for lowering serum cholesterol which includes the step of administering to a patient in need of treatment a synergistic combination of pravastatin and aspirin as described above.

The method of the invention is particularly adapted for patients having one or more risk factors for a coronary and/or cerebrovascular event. Such risk factors include hypercholesterolemia, coronary artery disease (CAD), family history of coronary artery disease, hypertension, diabetes, cigarette smoking, cerebrovascular disease and/or male gender.

The method of the invention is particularly useful in inhibiting, preventing or reducing cardiovascular events such as coronary heart disease and related death including fatal myocardial infarction, non-fatal myocardial infarction, myocardial revascularization procedures including coronary artery bypass surgery (CABG) and angioplasty (PCTA) or ischemic stroke.

The combination of pravastatin and aspirin may be administered in a fixed dosage form such as a bilayered tablet as disclosed in U.S. Pat. No. 6,235,311 to Ullah et al, the disclosure of which is incorporated herein by reference, or other known fixed dosage forms. Alternatively, the pravastatin and aspirin may be administered separately, in separate dosage forms, albeit, preferably, at the same time.

### DETAILED DESCRIPTION OF THE INVENTION

The pharmaceutical combination of the invention which is a synergistic combination of pravastatin and aspirin is effective in preventing, reducing and/or treating elevated cholesterol levels (such as in hypercholesterolemia), atherosclerosis, cardiovascular events and disease including coronary events and cerebrovascular events, and coronary artery disease and/or cerebrovascular disease.

The terms “cardiovascular event(s)” and “cardiovascular disease” as employed herein refer to coronary and/or cerebrovascular event(s) and disease including non-fatal myocardial infarction including primary myocardial infarction and secondary myocardial infarction, fatal myocardial infarction, myocardial ischemia, angina pectoris (including unstable angina), congestive heart failure, sudden cardiac death, cerebral infarction, cerebral thrombosis, ischemic stroke, cerebral ischemia, syncope, transient ischemic attack, as well as myocardial revasculation procedures including coronary bypass surgery (CABG) and angioplasty (PTCA).

The term “coronary artery disease” (CAD) as employed herein refers to diseases including atherosclerosis of the coronary arteries, previous myocardial infarction, ischemia, angina pectoris and/or heart failure.

The term “cerebrovascular disease” as employed herein refers to diseases including atherosclerosis of the intracranial and/or extracranial arteries, cerebral infarction, cerebral thrombosis, cerebral ischemia, stroke, and/or transient ischemic attacks.

Aspirin will preferably be employed in the form of salicylic acid acetate also referred to as acetylsalicylic acid. In preferred embodiments, the aspirin will be buffered as described hereinafter.

The pharmaceutical composition of the invention in the form of a tablet or capsule will include aspirin in amounts from about 81 mg or higher, preferably 81 mg or 325 mg.

The aspirin for use in forming the pharmaceutical combination of the invention will preferably be in the form of granules having an average particle size within the range from about 10 μm to about 2 mm, more preferably from about 0.25 mm to about 1.0 mm.

The pharmaceutical combination of the invention will contain pravastatin in an amount of 40 mg and aspirin in an amount of 81 mg or higher, which is administered in single or divided doses on a per daily basis.

The buffering agents present in buffered aspirin may include conventional acid buffers such as calcium carbonate, magnesium oxide, magnesium carbonate, magnesium hydroxide, aluminum hydroxide, dihydroxyaluminum sodium carbonate, aluminum magnesium hydroxide sulfate or aluminum hydroxide magnesium carbonate co-dried gel, or mixtures of one or more thereof, in amounts as needed to insure that the aspirin will be sufficiently buffered to inhibit GI side effects. Thus, amounts of buffering agent within the range from about 10 to about 1000 mg, preferably from about 50 to about 500 mg will be employed depending upon the amount of aspirin present.

In carrying out the method of the present invention, the pharmaceutical composition of the invention containing the combination of pravastatin and aspirin may be administered to mammalian species, such as monkeys, dogs, cats, rats, humans, etc., and, as described hereinbefore, may be incorporated in a tablet or capsule. The above dosage forms will also include the necessary carrier material, excipient, lubricant, buffer, antibacterial, bulking agent (such as mannitol), anti-oxidants such as Vitamin C and Vitamin E, as well as Vitamin B₆, Vitamin B₁₂, folic acid, sodium bisulfite, and the like.

The dose administered must be adjusted according to age, weight and condition of the patient, as well as the route of administration, dosage form and regimen and the desired result.

The compositions described above may be administered in the dosage forms as described above in single or divided doses of one to four times daily.

Tablets of various sizes can be prepared, e.g., of about 2 to 2000 mg in total weight, containing the active substances in the ranges described above, with the remainder being a physiologically acceptable carrier of other materials according to accepted pharmaceutical practice. These tablets can, of course, be scored to provide for fractional doses in some cases. Gelatin capsules can be similarly formulated.

Some of the active substances described above form commonly known, pharmaceutically acceptable salts such as alkali metal and other common basic salts or acid addition salts and the like. References to the base substances are therefore intended to include those common salts known to be substantially equivalent to the parent compound.

The formulations as described above will be administered for a prolonged period, that is, for as long as the potential for cardiovascular events and disease including coronary artery disease and/or cerebrovascular disease remains or the symptoms continue. Sustained release forms of such formulations which may provide such amounts daily, biweekly, weekly, monthly and the like may also be employed. A dosing period of at least 10 days are required to achieve minimal benefit.

### BRIEF DESCRIPTION OF THE FIGURES

FIGS. 1 to 20 are graphs showing the mean cumulative incidences (that is events) (FIGS. 1, 3, 5, 7, 9, 11, 13, 15, 17 and 19) and hazards (FIGS. 2, 4, 6, 8, 10, 12, 14, 16, 18 and 20) by treatment arm obtained employing the synergistic combination of the invention that is pravastatin and aspirin (referred to as pravapirin), pravastatin alone, aspirin alone and placebo.

The following Examples represent preferred embodiments of the present invention.

### EXAMPLE

The following example demonstrates the synergy between pravastatin (40 mg) and aspirin (81 mg or higher).

This example shows that the effects of pravastatin and aspirin are synergistic (that is, super-additive) in the sense that the combination (hereafter called prava/ASA) is more effective than the sum of the separate effects of the agents when used for secondary prevention.

In this study 40 mg pravastatin and 81 mg aspirin and higher were employed.

The primary endpoint was (i) any cardiac event, including ischemic stroke, or death. Secondary endpoints were individual components of the primary endpoint: (ii) any cardiac event, excluding stroke, (iii) any myocardial infarction, (iv) ischemic stroke, and (v) death. Analyses of these secondary endpoints as opposed to the primary endpoint are subject to greater uncertainty because the numbers of events are smaller. Therefore, conclusions for the secondary endpoints are likely to be less compelling.

The five studies available for addressing the synergy between pravastatin and aspirin for secondary prevention are listed and described in Table 1. Patients in all five studies were randomized to pravastatin vs placebo. Aspirin use was not randomized and it varied by study, as indicated in Table 1. All patients in all five studies are included in the primary analysis.

Table 1 shows that there are study differences in the use of the two agents and of their combination. These may be due to random variability or to differences in patient populations. To take these differences into account, the role of patient characteristics was modeled, namely, the following covariates were considered: age, gender, smoking status, existence of coronary artery disease, and baseline values of LDL and HDL cholesterols, triglycerides, and diastolic and systolic blood pressures. In addition, the studies were conducted under different circumstances and by different investigators and even in different countries. Therefore, there may be additional study-specific differences that cannot be explained by differences in measured patient covariates. To allow for the possibility that treatment effects depend on study even after accounting for patient characteristics, Bayesian hierarchical modeling was used in which the study is one level of experimental unit and patient-within-study is a second level.

A brief introduction to Bayesian hierarchical modeling and a description of standard Bayesian hierarchical Cox proportional hazards model (Model 1) are set out below. An extension of the standard model that allows for treatment-specific time-varying hazards (Model 2) is also described. The results of analyses of the five studies are shown in Table 1 using both models. In addition, the sensitivity of the conclusions to the prior distribution of the parameter that regulates the extent of pooling of the various studies is addressed.

Model 1: Bayesian Hierarchical Analysis Using Cox Proportional Hazards Model

The goal of a Bayesian hierarchical analysis is to synthesize all available information. Developments of and applications of hierarchical models are carried out by Lindley and Smith (1972), Berger (1986), DuMouchel (1989), Skene and Wakefield (1990), George et al (1994), Gelman et al (1995), Stangl (1995, 1996), Smith et al (1996), Christiansen and Morris (1996), Qian et al (1996), DuMouchel et al (1996), Carlin and Louis (1997), Berry D A (1998), Berry S M (1998) and Stangl and Berry (2000), among others. Effects are random in the sense that each study has a distribution of patient outcomes that is regarded to be selected from a population of study distributions. Patients in one study contain direct information about that study's distribution but they also give indirect information about the population of study distributions.

Such “borrowing strength” is standard in the Bayesian approach. However, a distinct benefit over simple pooling of data is that the extent of “borrowing” is dictated by the data. If the results (such as the relative benefits of the treatments) are different from one study to the next then there is very little borrowing. In this case the resulting posterior probability distributions have large variances and the associated conclusions about drug effects and drug interactions are weak. On the other hand, if the results are similar in the various studies then there is greater borrowing. But even if a drug's effect is identical in all five studies there is less borrowing than when pooling the data from the five studies. The Bayesian hierarchical approach allows for study heterogeneity and borrows less than do approaches (such as Mantel-Haenszel) that assume study homogeneity. Therefore, Bayesian hierarchical modeling is more conservative than approaches where the studies are to be homogeneous.

The Cox proportional hazards model takes hazard H to be of the form

H(t)=λ₀(t)exp(Zβ),

where Z is the set of patient-specific covariates (listed above), β is the vector of regression coefficients and λ₀(t) is the baseline hazard function. Treatment and study are considered separately from covariates Z, as follows:

H(t)=λ₀(t)exp(Zβ+φ_{S}+γ_{T}),

where S is study and T is treatment. The five studies (S) are those listed in Table 1: CARE, LIPID, PLAC I, PLAC II and REGRESS. The four treatments (T) are placebo, aspirin (+placebo), pravastatin and prava/ASA. Studies with S=CARE and treatment arms with T=placebo by setting φ_{CARE}=0 and γ_{placebo}=0 are compared.

Hazards may change over time and so baseline hazard λ₀ is allowed to depend on the year of study. Where time t is in years, the following is taken:

λ₀(t)=θᵢ for i−1<t≦i, for i=1,2,3,4,5.

No assumption about relationships among the values of these constants is taken.

The prior distributions of the various parameters are taken to be essentially noninformative, in the sense that they represent little prior information and so they will have negligible influence on the final conclusions. However, for calculational purposes it is required that the prior distributions are proper. It is assumed that the prior distributions are as follows:

[θᵢ]˜Gamma(a,b),

[βᵢ]˜Normal(0, σ_{β}²),

[γ_{T}]˜Normal(0, σ_{γ}²),

where a=0.05, b=1 (that is, the distribution of θᵢ is exponential with mean 0.05), σ_{β}=10 and σ_{γ}=10.

Study parameters φₛ are regarded as arising from a normal population:

[φ_{S}]˜Normal(μ_{φ}, σ_{φ}²),

where μ_{φ} and σ_{φ} are unknown. Assumptions about the parameter σ_{φ} are critical because it measures the extent of heterogeneity among the studies. If σ_{φ} is concentrated near 0 then the patients in the various studies would in effect be regarded as exchangeable and the analysis would be equivalent to pooling the patients into one large study. If σ_{φ} is assumed to be large then there would be no borrowing; each study in effect stands on its own. It is important to let the results of the studies determine whether and to what extent the studies can be pooled. The unknown parameters μ_{φ} and σ_{φ} are themselves random variables in the Bayesian approach and so they have probability distributions. It is assumed that:

[μ_{φ}]˜Normal(m_{φ}, s_{φ}²),

[σ_{φ}²]˜Gamma⁻¹(a_{φ}, b_{φ}),

and it is taken that m_{φ}=0, s_{φ}²=1. This distribution of μ_{φ} is essentially noninformative and so it plays a negligible role in the eventual conclusions.

Since assumptions about σ_{φ}, are critical, its assumed prior distribution is also critical. It is taken that α_{φ}=−½ and b_{φ}=1000. This distribution allows for both large (heterogeneity) and small (homogeneity) values of σ_{φ} and therefore it lets the empirical information dictate the extent of borrowing.

Assessing the probability of synergy is straightforward in Model 1 when using Markov chain Monte Carlo (MCMC) methods. Synergy means that γ_{prava/ASA}<γₚᵣₐᵥₐₛₜₐₜᵢₙ+γₐₛₚᵢᵣᵢₙ. During each iteration of MCMC, this synergy condition is assessed. Its posterior probability is the proportion of the iterations in which the condition holds. The estimated effect of prava/ASA that is beyond that of the sum of pravastatin and aspirin is γ_{prava/ASA}−(γₚᵣₐᵥₐₛₜₐₜᵢₙ+γₐₛₚᵢᵣᵢₙ) in the logarithmic scale.

Model 2: Extension to Treatment-Dependent Baseline Hazards

An underlying assumption of Model 1 above is that any modification of baseline hazard due to treatment is the same for all times t. Should it happen that aspirin is beneficial only in the first 2 years, say, while pravastatin has a later and potentially more durable benefit then this cannot be captured in Model 1. Therefore, in Model 2 the treatments are allowed to have time-varying effects while continuing to adopt a proportional hazards model for the covariates. Namely, the hazard in Model 2 is assumed to be

H(t)=λ_{T}(t)exp(Zβ+φ_{S}),

where λ_{T}(t) is the baseline hazard for treatment T. Where time t is in years:

λ_{T}(t)=θ_{T,i} for i−1<t≦i, for i=1,2,3,4,5.

The hazards for different treatments may differ, as will be seen in results below. The θ's have the same prior distribution as in Model 1:

[θ_{T,i}]˜Gamma(a,b)

where a=0.05 and b=1.

There is no perfect analog of γₐₛₚᵢᵣᵢₙ, γₚᵣₐᵥₐₛₜₐₜᵢₙ and γ_{prava/ASA} in Model 2 because hazards associated with treatment depend on the time period in question. To make for comparable interpretations of results across the two models, let Θ_{T} stand for the cumulative hazard for patients in treatment group T over the five-year period: Θ_{T}=θ_{T,1}+ . . . +θ_{T,5}. Define γ_{T} in Model 2 as follows:

γ_{T}=log[Θ_{T}/Θ_{placebo}],

where log is the natural logarithm. As in Model 1, γ_{placebo}=0. The analog of Θ_{T} in Model 1 is 5*θ_{T}. Using this notation, in both Model 1 and Model 2 the probability of being event-free at 5 years is

exp{−Θ_{placebo}exp(Zβ+φ_{S}+γ_{T})}.

With this definition of γ_{T}, the probability of synergy in Model 2 is calculated in the same way as in Model 1: namely, as the posterior probability that γ_{prava/ASA}<γₚᵣₐᵥₐₛₜₐₜᵢₙ+γₐₛₚᵢᵣᵢₙ. Again, this probability is found using MCMC.

Results

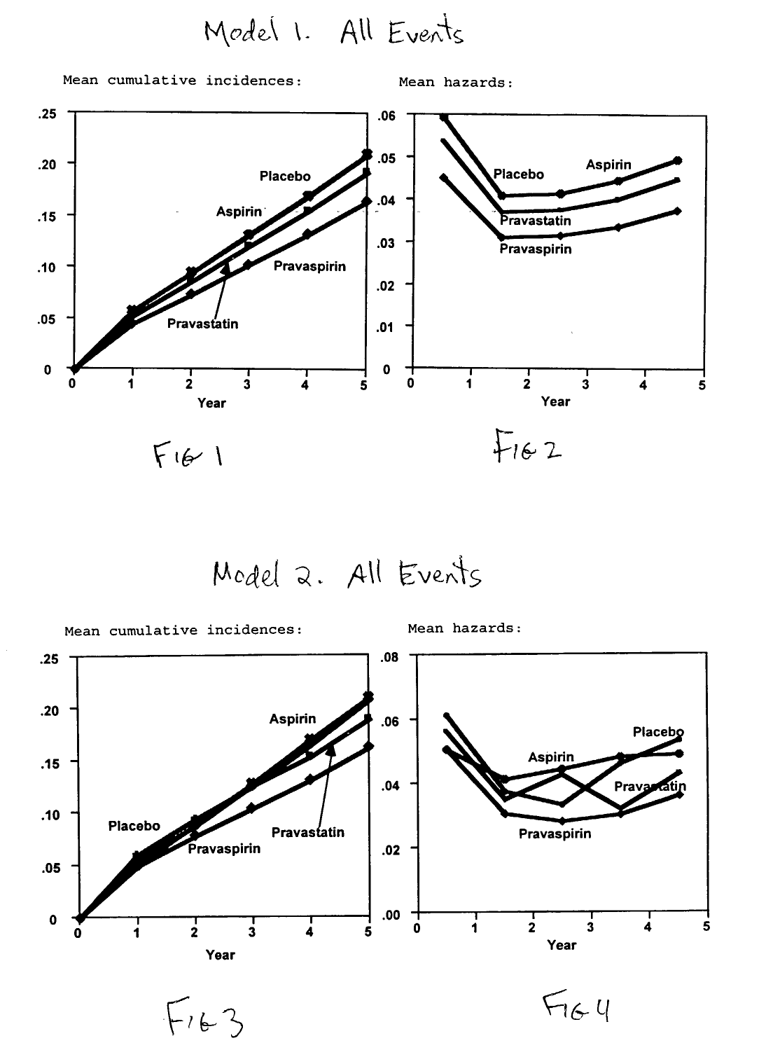

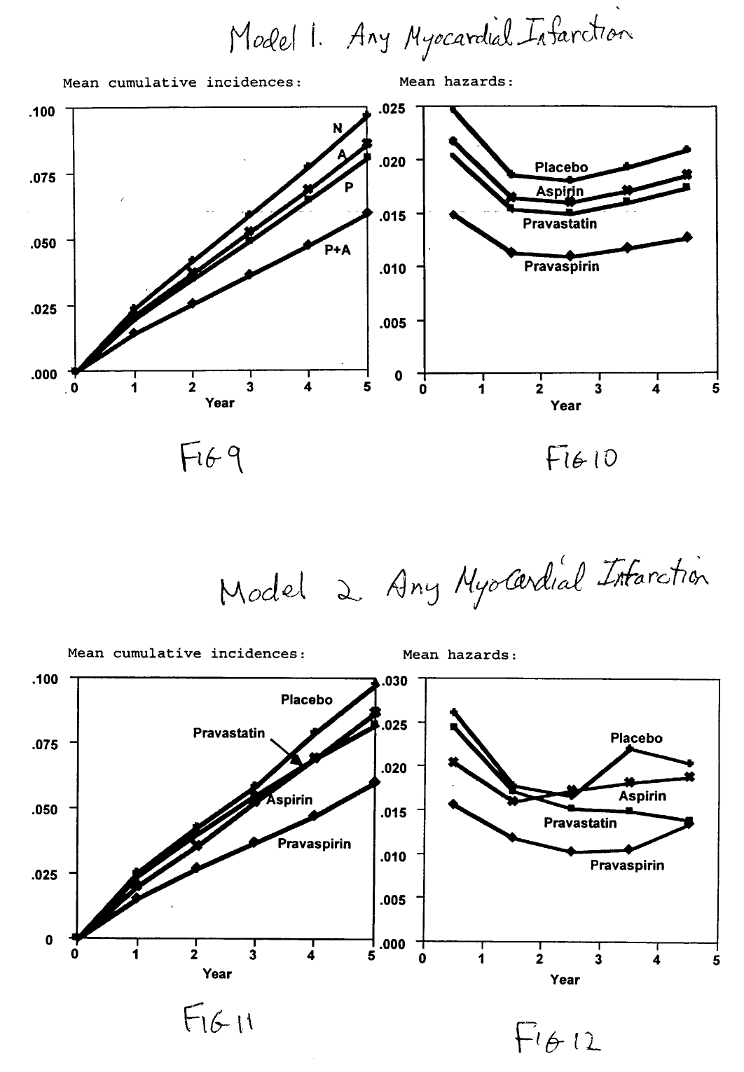

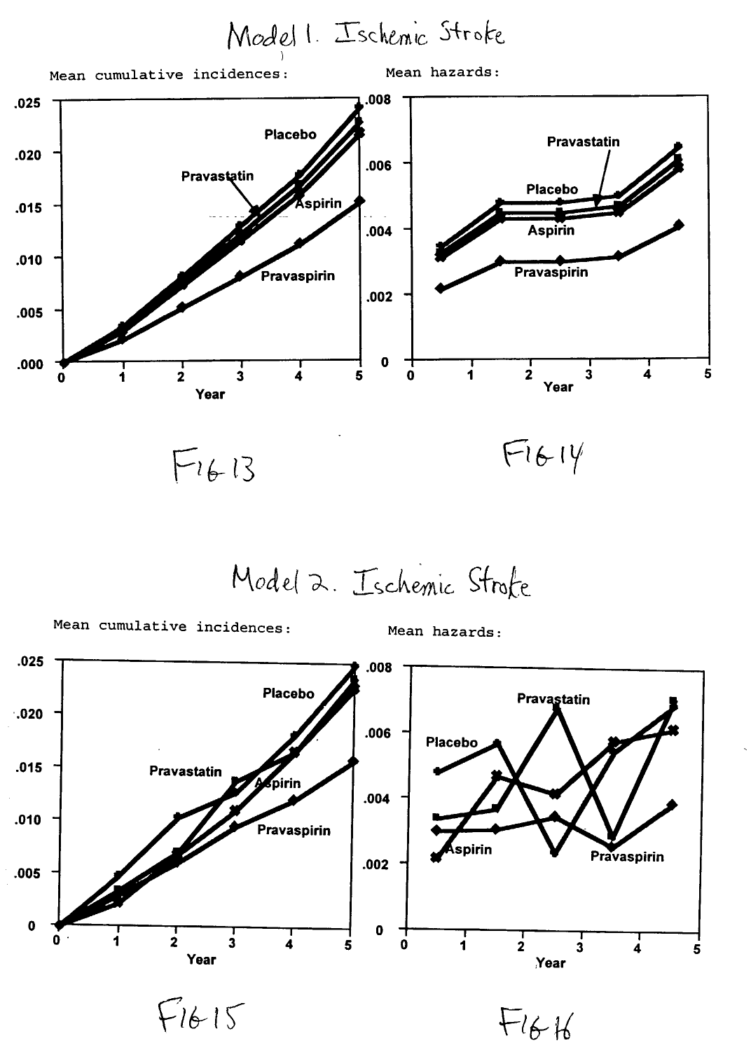

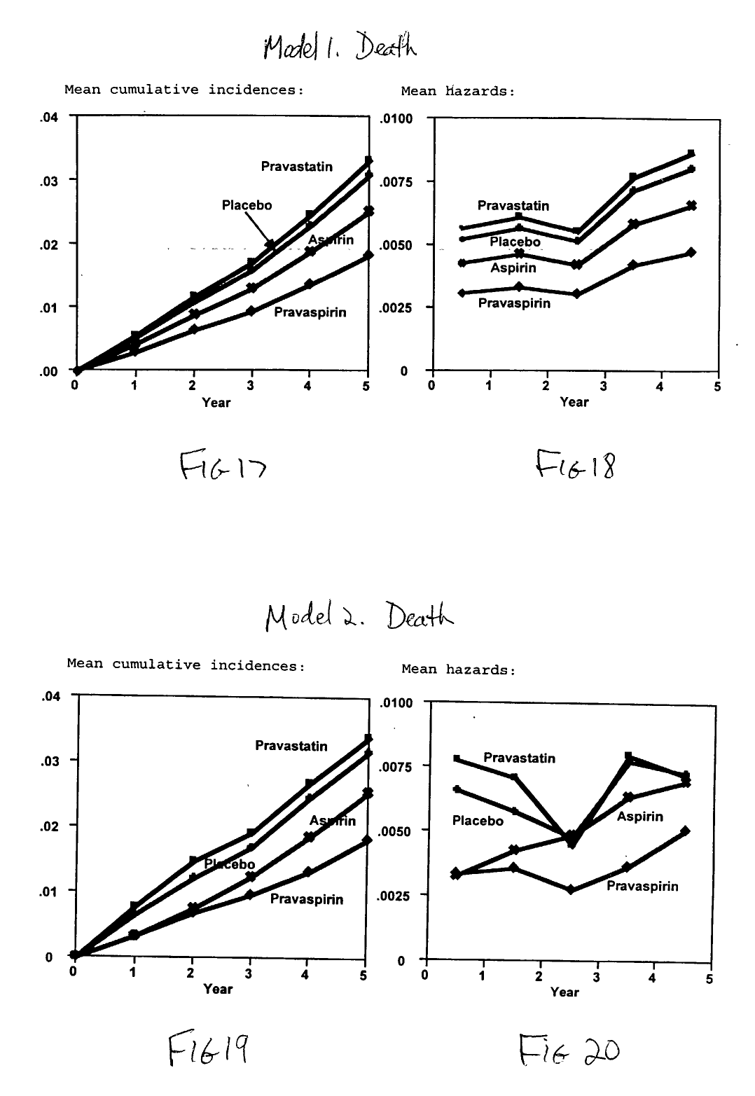

This section contains results for both Models 1 and 2. The results are very similar in both models, indicating that the relative benefits of the treatments are similar over the five years. The appendix contains summaries of the posterior distributions of the various parameters. The accompanying FIGS. 1 to 20 show the mean cumulative incidences and hazards by treatment arm. Baseline hazard rates are for a study participant who has average covariates, is female and did not have coronary artery disease at entry.

As shown in the appendix and FIGS. 1 to 4, for the primary endpoint (“All events” means all cardiac events, including ischemic strokes, and death) the aspirin+placebo and the placebo alone groups perform essentially the same. This is evident when using either of Models 1 and 2. However, patients taking both aspirin and pravastatin benefited greatly in comparison with placebo alone. Since approximately half the patients who took aspirin also received pravastatin, there was an overall benefit for aspirin (namely, the simple average of the aspirin and prava/ASA groups), but it was restricted to those who also received pravastatin.

The posterior means of the coefficients γ_{T} in Model 1 are 0, −0.0022, −0.0985, −0.2725, for T=placebo, placebo+aspirin, pravastatin, and prava/ASA. In the logarithmic scale the estimated effect of prava/ASA is −0.2725 while the sum of effects of pravastatin and aspirin is −0.0022−0.0985=−0.1007. Therefore, the estimated synergistic effect is −0.2725−(−0.1007)=−0.1718. The estimates of the various parameters are not independent and so the variance of the differences is not the sum of the variances of the individual estimates. However, as indicated above, the posterior probability that γ_{prava/ASA}<γₚᵣₐᵥₐₛₜₐₜᵢₙ+γₐₛₚᵢᵣᵢₙ can be found using MCMC. Namely, this probability is 0.983 (standard error 0.002, based on 5000 simulations).

Model 2 results show the changing estimated hazards over time and by treatment arm. The event rates of about 5-6% in the first year drop to 3-4% in the second year, gradually increasing thereafter. The hazard estimates by treatment arm evince some variability over time, but the estimated prava/ASA event rate is smallest of all treatment arms in each of the five years. The probability of synergy using Model 2 is 0.985, which is essentially the same as in Model 1.

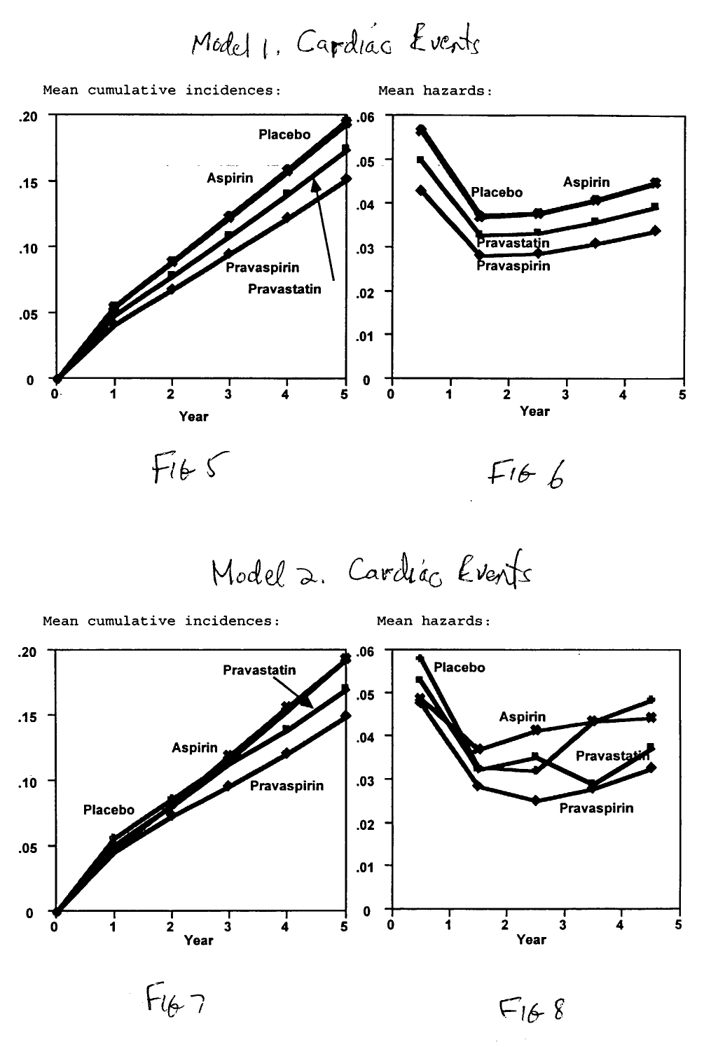

The results of Models 1 and 2 for the secondary endpoints (which are various components of the primary endpoint) are also shown in the appendix and FIGS. 5 to 20. There is more variability in estimated hazard and incidence rates for the individual endpoints because the numbers of events are smaller. This is especially so for stroke and death. However, the estimates evince the same general pattern as for the primary endpoint. In particular, the cumulative incidence rates are always smallest in the prava/ASA group. (Actually, this statement is also true for hazards, except for stroke in Model 2: aspirin has the lowest estimated event rate in year 1 and placebo has the lowest estimated event rate in year 3. However, the numbers of strokes are very small—see Table 1. Moreover, the cumulative incidence rates very strongly favor prava/ASA even in this case.)

Table 2 summarizes the results given in the appendix. It shows the remarkable consistency of the results. The probability of synergy is greater than 98% for the primary endpoint and it is greater than 90% for all the secondary endpoints.

Table 3 shows sensitivity results in which the prior distribution of the critical parameter σ_{φ}, is varied. The probability of synergy is consistently greater than 97%.

Table 4 shows the results for particular subsets. Again, the results are remarkably consistent. When dividing a data set in two and analyzing the pieces separately, one expects smaller posterior probabilities because this depends on posterior variance which depends on sample size. Indeed, the average probability dropped. But it did not drop much. The fact that the probability of synergy is about 86% for patients <65 years old and 98% for those ≧65 means that these two subsets provide independent confirmation that the two agents are synergistic. The smallest probability of synergy (58% for females) is still greater than 50%, which means that the estimated effects of prava/ASA are greater than the sums of the individual effects of pravastatin and aspirin even for this subset.

TABLE 2
Probability of synergy between pravastatin and aspirin for
the primary (in bold-faced type) and various secondary endpoints.

| | Probability of synergy | |
|---|---|---|
| Endpoint | Model 1 | Model 2 |

| | | |
|---|---|---|
| All events | 0.983 | 0.985 |
| Cardiac events | 0.945 | 0.947 |
| Any myocardial infarction | 0.911 | 0.923 |
| Ischemic stroke | 0.924 | 0.906 |
| Death | 0.997 | 0.997 |

TABLE 3
Sensitivity with respect to prior distribution of σ_{φ},.
Model 1 results for Endpoint = All events.

| | Probability of synergy between |
|---|---|
| Prior distribution | pravastatin and aspirin |
| Gamma⁻¹(−1/2, 1000) | 0.983 |
| Gamma⁻¹(1, 1) | 0.977 |
| Gamma⁻¹(3, 3) | 0.984 |
| Gamma⁻¹(5, 5) | 0.986 |
| Gamma⁻¹(10, 10) | 0.987 |
| Gamma⁻¹(50, 50) | 0.991 |

TABLE 4
Subset analyses.
Model 1 results for Endpoint = All events.

| | Probability of synergy between |
|---|---|
| Subset | pravastatin and aspirin |
| All patients | 0.983 |
| Females | 0.578 |
| Males | 0.988 |
| Age ≧ 65 | 0.985 |
| Age < 65 | 0.856 |

Conclusion

It has been shown that the effects of pravastatin and aspirin are synergistic in the sense that administering them in combination reduces event rates greater than would be expected by the addition of their separate effects. Analyses has been adjusted for differences in patient populations, for differences in study effects, and for the possibility that hazard rates are time-varying depending on treatment.

In addition, the conclusion of synergy holds separately for cardiac events, for myocardial infarctions, for ischemic strokes and for deaths. Moreover, it holds for both younger patients (<65 years old) and older patients. And it holds for both women and men.

### REFERENCES

Berger J O (1986). Statistical Decision Theory and Bayesian Analysis, 2nd ed., Springer-Verlag, N.Y.

Berry D A (1998). Benefits and risks of screening mammography for women in their forties: A statistical appraisal. Journal of the National Cancer Institute 90:1431-1439.

Berry S M (1998). Understanding and testing for heterogeneity across 2×2 tables: Application to meta-analysis. Statistics in Medicine 17:2353-2369.

Carlin B, Louis T (1997). Bayes and Empirical Bayes Methods. Chapman and Hall, London.

Christiansen C L, Morris C N (1996). Fitting and Checking a Two-Level Poisson Model: Modeling Patient Mortality Rates in Heart Transplant Patients. In: Bayesian Biostatistics. 467-501. Marcel Dekker, N.Y. (Ed: Berry D A, Stangl D K)

DuMouchel W (1989). Bayesian Metaanalysis. In: Statistical Methodology in the Pharmaceutical Sciences. 509-529. Marcel Dekker, N.Y. (Ed: D A Berry)

DuMouchel W, Waternaux C, Kinney D (1996). Hierarchical Bayesian Linear Models for Assessing the Effect of Extreme Cold Weather on Schizophrenic Births. In: Bayesian Biostatistics. 451-465. Marcel Dekker, N.Y. (Ed: Berry D A, Stangl D K)

Gelman A, Carlin J B, Stern H S, Rubin D B (1995). Bayesian Data Analysis. Chapman & Hall, London.

George S L, Li C C, Berry D A, Green M R (1994). Stopping a clinical trial early: Frequentist and Bayesian approaches applied to a CALGB trial in non-small cell lung cancer. Statistics in Medicine 13:1313-1327.

Lindley D V, Smith A F M (1972). Bayes estimates for the linear model (with discussion), Journal of the Royal Statistical Association, Series B 34:1-41.

Qian J, Stangl D K, George S (1996). A Weibull Model for Survival Data: Using Prediction to Decide When to Stop a Clinical Trial. In: Bayesian Biostatistics. 187-205. Marcel Dekker, New York. (Ed: Berry D A, Stangl D K)

Skene A M, Wakefield J C (1990). Hierarchical models for multicentre binary response studies. Statistics in Medicine 9:919-929.

Smith T C, Spiegelhalter D J, Parmar M K B (1996). Bayesian Meta-Analysis of Randomized Trials Using Graphical Models and BUGS. In: Bayesian Biostatistics. 411-427. Marcel Dekker, New York. (Ed: Berry D A, Stangl D K)

Stangl D (1995). Prediction and decision making using Bayesian hierarchical survival models. Statistics in Medicine 14: 2173-2190.

Stangl D K (1996). Hierarchical Analysis of Continuous-Time Survival Models. In: Bayesian Biostatistics. 429-450. Marcel Dekker, New York. (Ed: Berry D A, Stangl D K)

Stangl D K, Berry D A (2000). Meta-Analysis in Medicine and Health Policy. Marcel Dekker, New York.

[image]

[image]

[image]

[image]

What is claimed is:

1. A method for preventing, inhibiting or reducing the risk of onset of a cardiovascular event or for lowering serum cholesterol, which comprises administering to a patient in need of treatment a synergistic combination of pravastatin and aspirin.

2. The method as defined in claim 1 wherein the synergistic combination comprises 40 mg pravastatin and at least 81 mg aspirin administered per day.

3. The method as defined in claim 1 wherein the synergistic combination comprises 40 mg pravastatin and 81 mg aspirin or 325 mg aspirin administered per day.

4. The method as defined in claim 1 wherein the aspirin is buffered aspirin.

5. The method as defined in claim 1 wherein the patient to be treated has one or more risk factors.

6. The method as defined in claim 5 wherein the risk factors are one or more of hypercholesterolemia, coronary artery disease, family history of coronary artery disease, hypertension, diabetes, cigarette smoking, cerebrovascular disease and male gender.

7. The method as defined in claim 1 wherein the cardiovascular event is a primary myocardial infarction, a secondary myocardial infarction, angina pectoris, unstable angina, congestive heart failure, sudden cardiac death, cerebral infarction, syncope or transient ischemic attack.

8. The method as defined in claim 1 wherein the cardiovascular event is coronary heart disease and related death, fatal myocardial infarction, non-fatal myocardial infarction, myocardial revascularization procedures, coronary artery bypass surgery (CABG), PTCA (angioplasty), or ischemic stroke.

9. The method as defined in claim 8 wherein the synergistic combination employed is 40 mg pravastatin and 81 mg aspirin.

10. The method as defined in claim 8 wherein the synergistic combination employed is 40 mg pravastatin and 325 mg aspirin.

11. A synergistic combination comprising 40 mg pravastatin and 81 mg aspirin or 40 mg pravastatin and 325 mg aspirin.

12. A pharmaceutical composition comprising the synergistic combination as defined in claim 11 and a pharmaceutical acceptable carrier therefor.
